SEQUENCE LISTING (2 sequences)

SEQ ID NO: 1 - protein, 108 aa, Artificial Sequence
  Synthetic Construct
DIVMTQSHKFMSTSIGARVSITCKASQDVRTAVAWYQQKPGQSPKLLIYSASYRYTGVPDRFTGSGSGTDFTFTISSVQAEDLAVYYCQQHYGTPPWTFGGGTKLEIK

SEQ ID NO: 2 - protein, 117 aa, Artificial Sequence
  Synthetic Construct
EVQLVESGGGLVKPGGSLKLSCEASRFTFSSYAMSWVRQTPEKRLEWVAAISGGGRYTYYPDSMKGRFTISRDNAKNFLYLQMSSLRSEDTAMYYCARHYDGYLDYWGQGTTLTVSS

(19) United States
(12) Patent Application Publication
(10) Pub. No.: US 2024/0408135 A1
(43) Pub. Date: Dec. 12, 2024

(54) METHODS FOR TREATING GRAFT VERSUS HOST DISEASE

(71) Applicant: THE GENERAL HOSPITAL CORPORATION, BOSTON, MA (US)

(72) Inventors: Xinhui WANG, Boston, MA (US); Soldano FERRONE, Boston, MA (US); Joseph H. SCHWAB, Boston, MA (US); Yi-Bin CHEN, Boston, MA (US); Zachariah DEFILIPP, Boston, MA (US)

(21) Appl. No.: 18/290,114

(22) PCT Filed: May 11, 2022
(86) PCT No.: PCT/US2022/028830

§ 371 Date: Nov. 9, 2023

Related U.S. Application Data
(60) Provisional application No. 63/187,128, filed on May 11, 2021.

(51) Int. Cl. A61K 35/17; A61K 39/00; A61P 37/06; C07K 14/705; C07K 14/725

(52) U.S. Cl. CPC A61K 35/17; A61K 39/4611; A61K 39/4631; A61K 39/46434; A61K 39/464411; A61P 37/06; C07K 14/7051; C07K 14/70517; A61K 2239/38; C07K 2317/53; C07K 2317/56

(57) ABSTRACT

The invention features a method for treating graft versus host disease in a human, including administering to said human a therapeutically effective amount of a cell including a chimeric antigen receptor (CAR) which is specifically directed against an immune checkpoint molecule.

### CROSS-REFERENCE TO RELATED APPLICATIONS

This application claims benefit of U.S. Provisional Application No. 63/187,128, filed on May 11, 2021, the contents of which are incorporated herein by reference in their entirety.

### SEQUENCE LISTING

The instant application contains a Sequence Listing which has been submitted electronically in ASCII format and is hereby incorporated by reference in its entirety. The ASCII copy, created on May 11, 2022, is named 51412-007WO2_Sequence_Listing_5_11_22_ST25 and is 2,406 bytes in size.

### BACKGROUND OF THE INVENTION

The invention relates to treating graft versus host disease.

Allogeneic hematopoietic stem cell transplantation (HSCT) is a potentially curative therapy for many hematological malignant and non-malignant diseases. Acute GVHD is a common complication after allogeneic HSCT and remains a major cause of morbidity with 30-60% of recipients requiring systemic therapy. In addition, acute GVHD is the leading cause of early mortality after HSCT, and thus represents a significant obstacle to successful HSCT outcomes. The primary organs involved in acute GVHD include the skin, gastrointestinal tract and liver. Initial therapy for acute GVHD has remained high-dose systemic corticosteroids for the last three decades, yet over 50% of patients treated with steroids will require additional therapy either due to progressive disease or inadequate response. Historically, improved methods of acute GVHD prevention or treatment have resulted in unacceptable cumulative immunosuppression, resulting in increased rates of disease relapse or opportunistic infection. Recently, novel agents with less broad immunosuppressive properties have suggested the ability to better prevent or treat acute GVHD without causing negative effects, yet outcomes after HSCT remain suboptimal. Therefore, improved methods of acute GVHD prevention and effective treatments of established acute GVHD remain significant unmet needs to improve overall outcomes after HSCT.

### SUMMARY OF THE INVENTION

In one aspect, the invention features a method for treating graft versus host disease (GVHD) in a human, including administering to the human a therapeutically effective amount of cells each of which includes a chimeric antigen receptor (CAR) which is specifically directed against an immune checkpoint molecule. In embodiments, the cells are produced using immune cells obtained from a donor of cells or tissue which is to be transplanted. In other embodiments, GVHD is acute or chronic GVHD. In still other embodiments, the immune checkpoint molecule is B7-H3, PD-1/PD-L1, TIM-3, or B7-H4. In other embodiments, the cells are T lymphocytes or NK cells. In other embodiments, the CAR is specifically directed against B7-H3. In embodiments of the B7-H3 CAR, the CAR includes variable regions of the heavy and light chains of the 376.96 mAb. In embodiments, the CAR includes a human CD8a hinge and transmembrane domain, CD28 or 4-1BB intracellular costimulatory domains, and CD3z intracellular signaling domain. In embodiments, the cells are administered intravenously. In embodiments, the cells are administered within 24 hours after a transplantation procedure.

In another aspect, the invention features a method of treating allogeneic tissue that is to be transplanted into a human, including contacting the tissue with cells each of which includes a chimeric antigen receptor (CAR) which is specifically directed against an immune checkpoint molecule. In embodiments, the allogeneic tissue includes cells. In embodiments, the immune checkpoint molecule is B7-H3, PD-1/PD-L1, TIM-3, or B7-H4. In other embodiments, the cells are T lymphocytes or NK cells. In other embodiments, the CAR is specifically directed against B7-H3.

In another aspect, the invention features a method for treating graft versus leukemia (GvL) in a human, including administering to the human a therapeutically effective amount of cells each of which includes a chimeric antigen receptor (CAR) which is specifically directed against an immune checkpoint molecule. In embodiments, the cells are produced using immune cells obtained from a donor of cells or tissue which is to be transplanted. In still other embodiments, the immune checkpoint molecule is B7-H3, PD-1/PD-L1, TIM-3, or B7-H4. In other embodiments, the cells are T lymphocytes or NK cells. In other embodiments, the CAR is specifically directed against B7-H3. In embodiments of the B7-H3 CAR, the CAR includes variable regions of the heavy and light chains of the 376.96 mAb. In embodiments, the CAR includes a human CD8a hinge and transmembrane domain, CD28 or 4-1BB intracellular costimulatory domains, and CD3z intracellular signaling domain. In embodiments, the cells are administered intravenously. In embodiments, the cells are administered within 24 hours after a transplantation procedure.

Unlike the currently available or investigational GVHD prophylaxis and management using immune-suppressive approaches to suppress immune system or T cell depletion, the methods described herein provide a targeted approach with selective elimination of alloreactive B7-H3+ T cells using B7-H3 CAR T cells. Due to sparing other immune cells, especially T cells, B7-H3 CAR T cell-therapy described herein provides several advantages including i) better preserving the efficacy of allogeneic hematopoietic stem cell transplantation (HSCT) such as graft versus leukemia (GvL) effect while effectively preventing/treating GVHD and ii) reducing the risk of viral infections, a major complication of HSCT during the period of immune suppression that follows the procedure and a leading cause of morbidity and mortality after allogeneic hematopoietic stem cell transplantation.

Other features and advantages of the invention will be apparent from the following description of the preferred embodiments thereof, and from the claims.

### BRIEF DESCRIPTION OF THE DRAWINGS

FIG. 1 shows B7-H3 CAR T cells prevented GVHD.

FIG. 2 shows long-term survival of B7-H3 CAR T.

FIG. 3 shows a schema of a human mouse model.

FIG. 4 shows that B7-H3 CAR T cells prevented GVHD.

FIG. 5 shows long term survival of human PBMC grafts

FIG. 6 shows B7-H3 is slightly expressed on in vitro activated T lymphocytes at day 5.

FIG. 7 shows B7-H3 is slightly expressed on in vitro activated T lymphocytes at day 8.

FIG. 8 shows B7-H3 is expressed on human CD3+ T cells after transplantation.

FIG. 9 shows B7-H3 CAR-T cells prevented GVHD in human PBMC engrafted NSG-(Kb Db) null (IAnull) mice.

FIG. 10 shows a small population of activated T cells expresses B7-H3 detected in a mixed lymphocyte reaction.

FIG. 11 shows that B7-H3 is expressed on peripheral circulating T cells and infiltrated T cells in the liver following humanization of immunodeficient NSG mice (xenogeneic GVHD model).

FIG. 12 shows that B7-H3 is expressed on peripheral circulating T cells in allogeneic mouse GVHD models.

FIG. 13 shows treatment of acute GVHD by B7-H3 CAR T cells in human PBMC engrafted NSG mice.

### DETAILED DESCRIPTION OF THE INVENTION

Described below is a highly effective and long-lasting approach to treating GVHD without immune-suppressive side effects by targeting immune checkpoint molecules (such as B7-H3, PD-1/PD-L1, TIM-3, and B7-H4) using CAR T cell therapy (for example, by administering donor-derived B7-H3 CAR T cells). As used herein, the term “CAR” or alternatively a “chimeric antigen receptor” refers to a recombinant polypeptide including at least an extracellular binding domain, a transmembrane domain and a cytoplasmic signaling domain including a functional signaling domain derived from a stimulatory molecule as described below. In one aspect, the CAR includes a human CD8a hinge and transmembrane domain, CD28 or 4-1BB intracellular costimulatory domains, and CD3z intracellular signaling domain. Such CARs are expressed in a variety of immune cells such as T lymphocytes or NK cells. Such cells may be autologous or allogeneic.

In one example, patients undergoing an allogeneic transplant procedure are administered any of the CAR T cells (alone or in combination) described herein according to standard procedures. Preferably, such CAR T cells are produced, according to standard methods, using immune cells obtained from the donor of the cells or tissue which is to be transplanted. In some embodiments, CAR Ts may be manufactured by collecting immune cells from the recipient of a transplant as well. CAR T cells are subsequently administered in a therapeutically effective amount, for example, to prevent development of, or alleviate to some extent, one or more of the signs or symptoms of GVHD, thereby treating GVHD. Such treatment results, for example, in reducing the frequency or severity of at least one sign or symptom of GVHD experienced by a subject receiving such treatment (e.g., a human).

### CAR T Cells

CAR T cells, for example, directed against B7-H3, PD-1/PD-L1, TIM-3, and B7-H4 are produced according to standard methods. For example, B7-H3 CAR T cells are described in U.S. Pat. No. 10,519,214, Du et al., Cancer Cell. 35 (2): 221-237 (2019), and Zhang et al., Mol Cancer Ther. 20 (3): 577-588 (2021). PD-1/PD-L1 CAR T cells are described, for example, in Yang et al., Mol Ther Oncolytics. 26:17-571-585 (2020). TIM-3 CAR T cells are described, for example, in Lee et al., Mol Cancer Ther. 20(9): 1702-1712 (2021) and He et al., Blood. 135 (10): 713-723 (2020). And CAR T cells directed against B7-H4 are described, for example, in Smith et al., Mol Ther. 24 (11): 1987-1999 (2016) and Saha et al. 4 (19): e127716 (2019), and Veenstra et al., Blood. 125 (21): 3335-46 (2015).

### Administration

CAR T cells described herein may be administered at a dosage of 10⁴ to 10⁹ cells/kg body weight. in some instances 10⁵ to 10⁶ cells/kg body weight. CAR T cells, for example, may also be administered multiple times at these dosages. The cells can be administered by using infusion techniques that are commonly known in immunotherapy (see, e.g., Rosenberg et al., New Eng. J. of Med. 319:1676, 1988). Regarding timing of administration, such cells are administered, for example, within 24 hours after transplantation and may be repeated on a daily or weekly basis generally not exceeding three months following transplantation.

Other immune cells such as NK cells may be made and administered in the methods described herein.

The following example using B7-H3 CAR T cells illustrates the methods described herein and is applicable to using CAR T cell therapy in treating GVHD directed against additional immune checkpoint molecules (such as PD-1/PD-L1, TIM-3, and B7-H4).

### B7-H3 CAR T Cells Prevented GVHD in Humanized Mice

### Methods

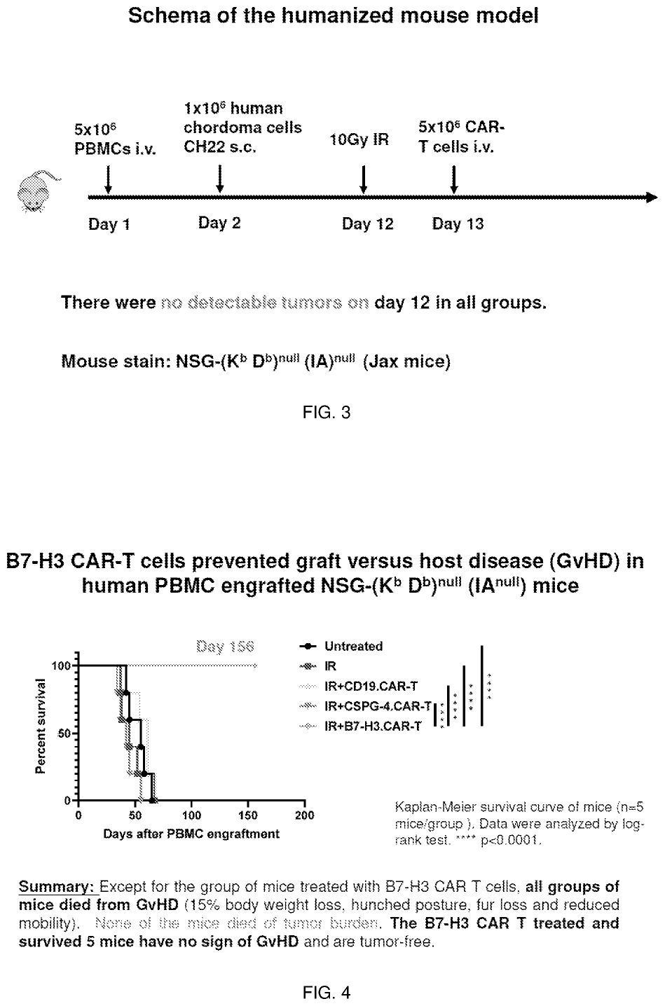

One experimental design was to evaluate if there is an enhanced immune response when using combined radiation therapy (IR) and the immune checkpoint B7-H3 chimeric antigen receptor (CAR) T cells in a humanized mouse model.
Mice: NSG-(Kb Db) null (IA) null (Jax mice) (n=5/group) (FIG. 3)
Timeline:
   Day 0: humanizing all mice by i.v. injection of human PBMC (5×10⁶/mouse)
   Day 2: Inoculation of human chordoma cells CH22 s.c (1×10⁶/mouse)
   Day 12: local irradiation (IR, 10 Gy) on tumor cell inoculated area
   Day 13: injection of B7-H3 or control CAR T cells 5×10⁶/mouse)

### Making B7-H3 CAR T Cells

The anti-B7-H3 CAR construct was derived from the single-chain variable fragment (scFv) of the B7-H3-specific 376.96 mAb. B7-H3 CAR T cells were generated as described in Zhang et al. (Mol Cancer Ther 2021 Mar. 20 (3): 577-588) as follows.

Peripheral blood mononuclear cells (PBMCs) were isolated from normal human donor blood (Research Blood Components, MA) with Lymphoprep (Stem cell Technologies). On day 0, the PBMCs (1×10⁶/well) were activated in a non-treated 24-well cell culture plate (#351147,Corning) pre-coated with 1 μg/mL CD3 (clone OKT3, Miltenyi Biotec) and 3 μg/mL CD28 antibodies (clone CD28.2, BD Biosciences) in the complete medium (45% RPMI1640 and 45% Click's medium [Irvine Scientific, CA], 10% FBS, 1% Penicillin and 1% Streptomycin [Corning]). On day 1, activated T cells were expanded by addition of IL-7 (10 ng/ml, PeproTech, NJ) and IL-15 (5 ng/ml, PeproTech) (CAR T medium). On day 2, the activated and expanded T cells were transferred to wells of 24-well plates that had been previously coated with RetroNectin (Takara Bio Inc., Shiga, Japan) and contained retroviral particles of the B7-H3 CAR construct (Du et al., Antitumor responses in the absence of toxicity in solid tumors by targeting B7-H3 via chimeric antigen receptor T cells. Cancer Cell 2019; 35:221-37. e8.). On day 4, to allow for their continued expansion, the transduced cells were collected and transferred to tissue culture-treated 24-well plates (Cat #353047 Corning) with each well containing 0.5 mL of the activated T cell suspension (5×10⁵ cells/well) and 1.5 mL of fresh CAR T medium. On day 6, an aliquot of transduced cells was analyzed for transduction efficiency and 50% CAR T spent medium was replaced with fresh medium, i.e., 50:50 (v./v.) old medium: new medium. On day 8, CAR T cells were counted and reseeded at 1×10⁶/well in 2 mL of fresh CAR T medium to further expand cells. On day 10, 50% spent medium was replaced with the fresh medium as done on day 6. On day 12-13, CAR T cells and non-transduced T (NT) cells grown at similar conditions were collected, aliquoted, and frozen for storage in a liquid nitrogen freezer for in vitro and in vivo experiments. See also U.S. Pat. No. 10,519,214 for a description of generating B7-H3 CAR T cells.

Accordingly, cells which include a B7-H3 CAR have the following:
a) a signal peptide;
b) a light chain variable region comprising the amino acid sequence: (SEQ ID NO:1)

DIVMTQSHKFMSTSIGARVSITCKASQDVRTAVAWYQQKPGQSPKLLIYS
ASYRYTGVPDRFTGSGSGTDFTFTISSVQAEDLAVYYCQQHYGTPPWTFG
GGTKLEIK;

c) a linker peptide;
d) a heavy chain variable region comprising the amino acid sequence: (SEQ ID NO:2)

EVQLVESGGGLVKPGGSLKLSCEASRFTFSSYAMSWVRQTPEKRLEWVAA
ISGGGRYTYYPDSMKGRFTISRDNAKNFLYLQMSSLRSEDTAMYYCARHY
DGYLDYWGQGTTLTVSS;

e) a CD8a hinge polypeptide;
f) a CD8a transmembrane domain;
g) a 4-1BB or CD28 costimulatory domain; and
h) a CD3Z signaling domain.

### Results

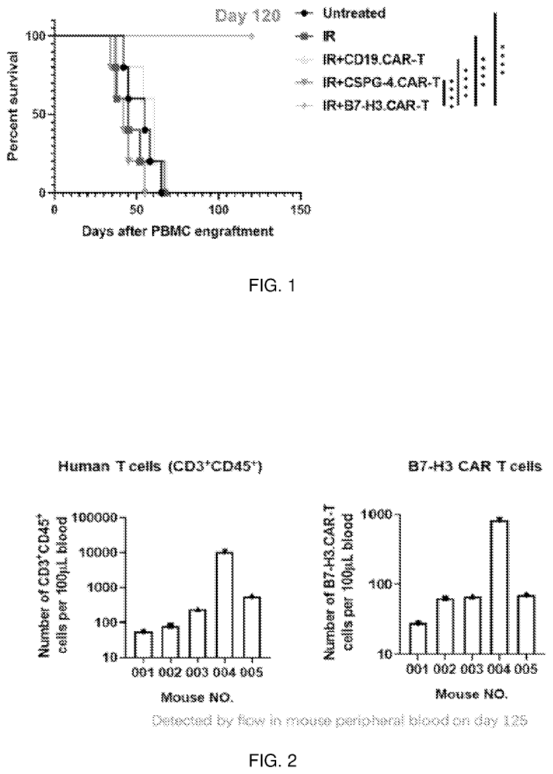

B7-H3 CAR T cells prevented GVHD in 100% humanized mice, while all other mice developed and died from GVHD (15% body weight loss, hunched posture, fur loss and reduced mobility) (FIG. 1 and FIG. 4).

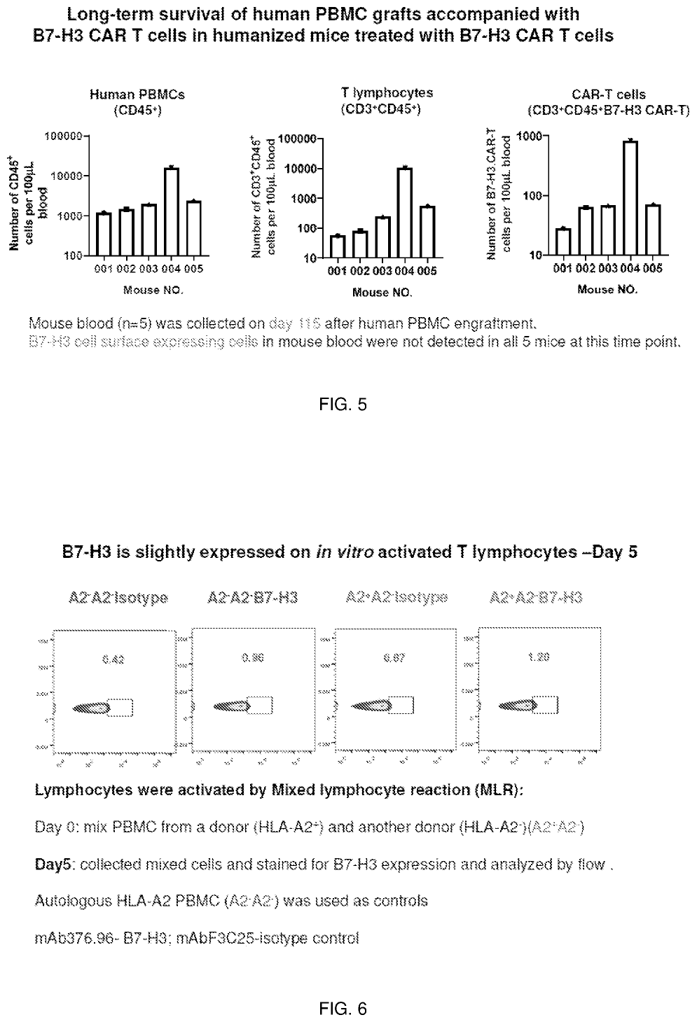

Long-term survival of human PBMC grafts including T lymphocytes was found in all B7-H3 CART cells treated humanized mice (FIG. 2 and FIG. 5).

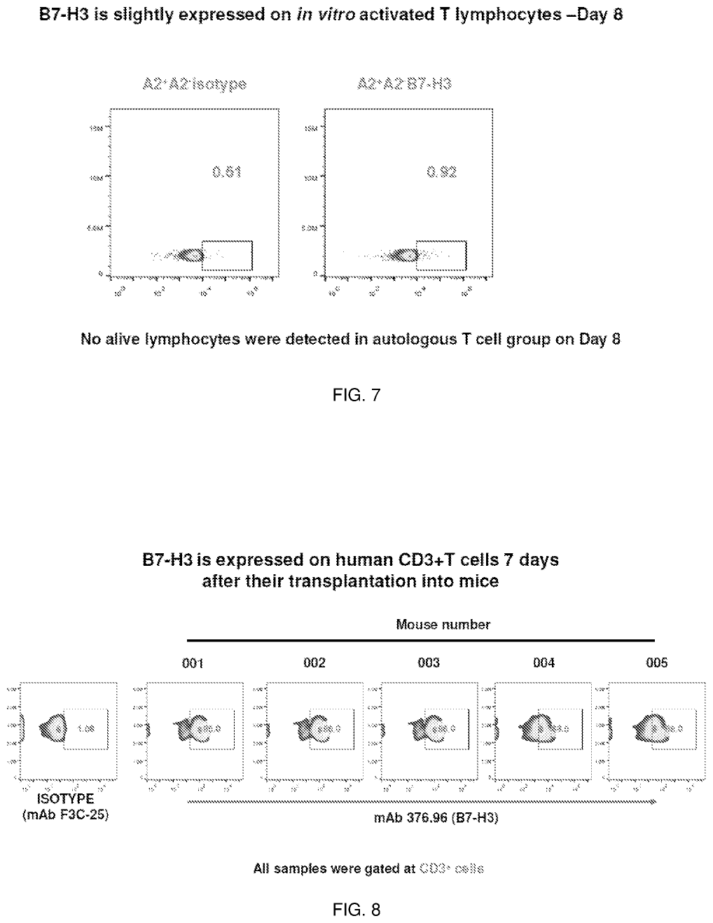

B7-H3 is slightly expressed on in vitro activated T lymphocytes (FIG. 6 and FIG. 7).

Long-term survival of B7-H3 CAR T cells was found along with the human PBMC grafts (FIG. 2).

B7-H3 is expressed on human T cells 7 days after their transplantation into mice (FIG. 8).

### Clinical Implication

Unlike the currently available or investigational GVHD prophylaxis and management using immune-suppressive approaches to suppress immune system or T cell depletion, our approach provides patients who undergo allo-HSCT by (1) preventing/treating GVHD via selective elimination of alloreactive B7-H3+ T cells using B7-H3 CAR T cells. Due to sparing other immune cells, especially T cells, B7-H3 CAR T cell-therapy shall better preserve the efficacy of allogeneic hematopoietic stem cell transplantation (HSCT) such as graft versus leukemia (GvL) effect or (2) reducing the risk of viral infections by selective elimination of alloreactive B7-H3+ T cells using B7-H3 CAR T cells, while keeping the immune system functional instead of being suppressed by immunosuppressive treatment or T cell depletion.

### B7-H3 CAR-T Cells Prevented GVHD in Human PBMC Engrafted NSG-(Kb db) Null (IAnull) Mice

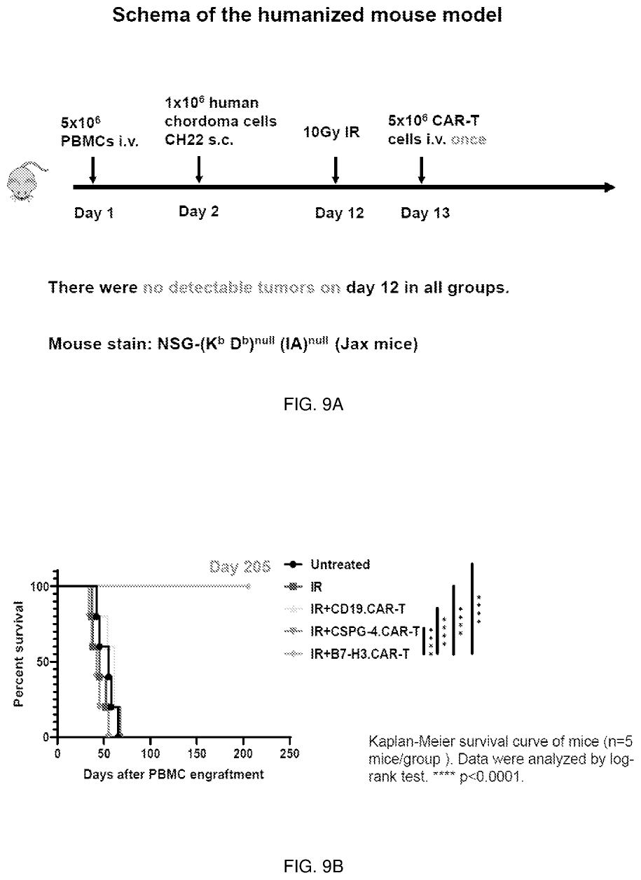

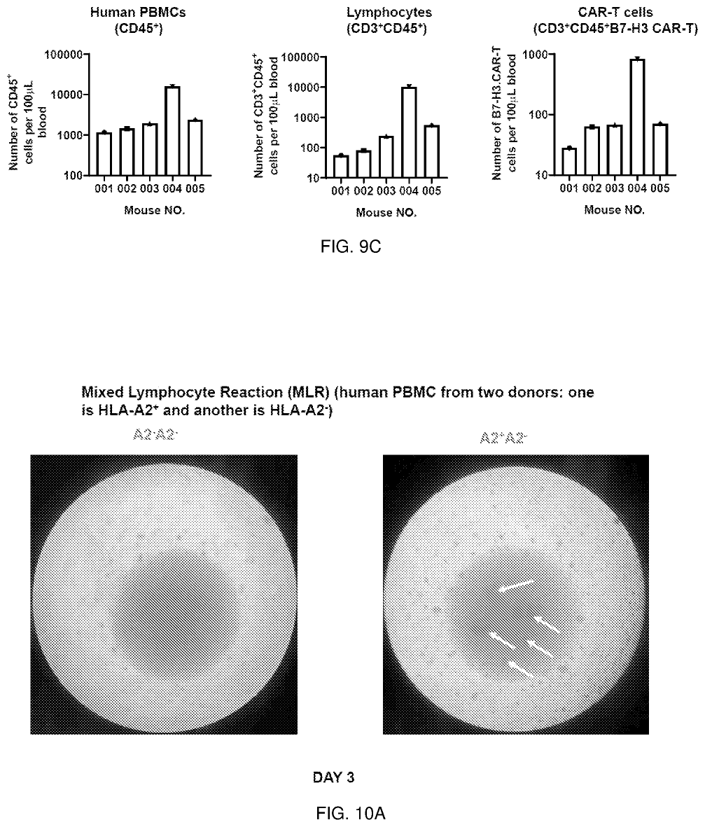

FIG. 9 shows that B7-H3 CAR-T cells prevented GVHD in human PBMC engrafted NSG-(Kb Db) null (IAnull) mice. To determine if B7-H3 CAR T therapy was bifunctional, i.e., could they abrogate the B7-H3 immune checkpoint and mediate lysis of targeted human chordoma cells CH22, the in vivo experiment was performed and the timeline of each procedure was outlined (FIG. 9A). The chordoma xenografts were not developed well or rejected due to prior implanted human immunity. Irradiation (IR) was delivered in residual tumor or tumor cells injected area of each mouse. Except for the group of mice treated with B7-H3 CAR T cells, all groups of mice died from GVHD (15% body weight loss, hunched posture, fur loss and reduced mobility). None of the mice died of tumor burden. The B7-H3 CAR T treated and survived 5 mice have no sign of GVHD and are tumor-free. CD19 is not expressed by CH22 cells. Tumor associated antigen chondroitin sulfate proteoglycan 4 (CSPG4) is expressed by CH22 cells, both were used as controls (FIG. 9B). In addition, long-term survival of human PBMC grafts (CD45+), CD45+CD3+ T cells as well as B7-H3 CAR T cells in humanized mice treated with B7-H3 CAR T cells were detected on day 115 after human PBMC engraftment. At this point, B7-H3 expressing T cells in mouse blood were not detected in all 5 mice (FIG. 9C). Long-term survival of engrafted human PBMC, T lymphocytes and B7-H3 CAR T cells in all B7-H3 CAR T cells treated humanized mice.

Based on the initial unexpected finding, we hypothesized that the immune checkpoint molecule B7-H3 is expressed upon T cell activation as a result of an immune-regulation response. These activated T cells expressing B7-H3 are possibly responsible for GVHD, and thus elimination of activated T cells by B7-H3 CAR T cells can potentially prevent/treat GVHD. To test this hypothesis, the following series of experiments were conducted.

### A Small Population of Activated T Cells Expresses B7-H3 Detected in Mixed Lymphocyte Reaction

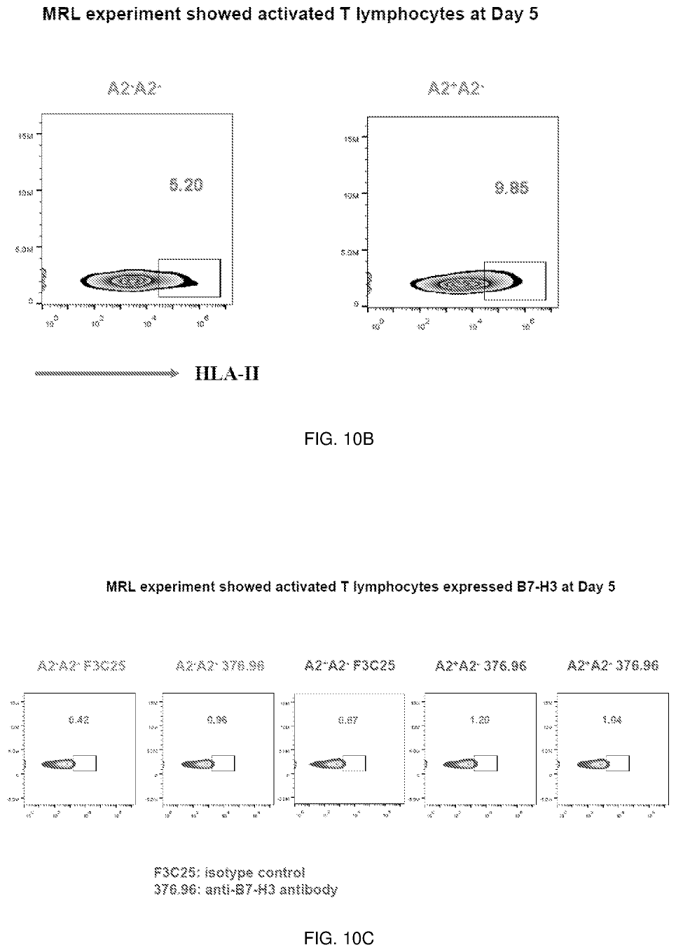

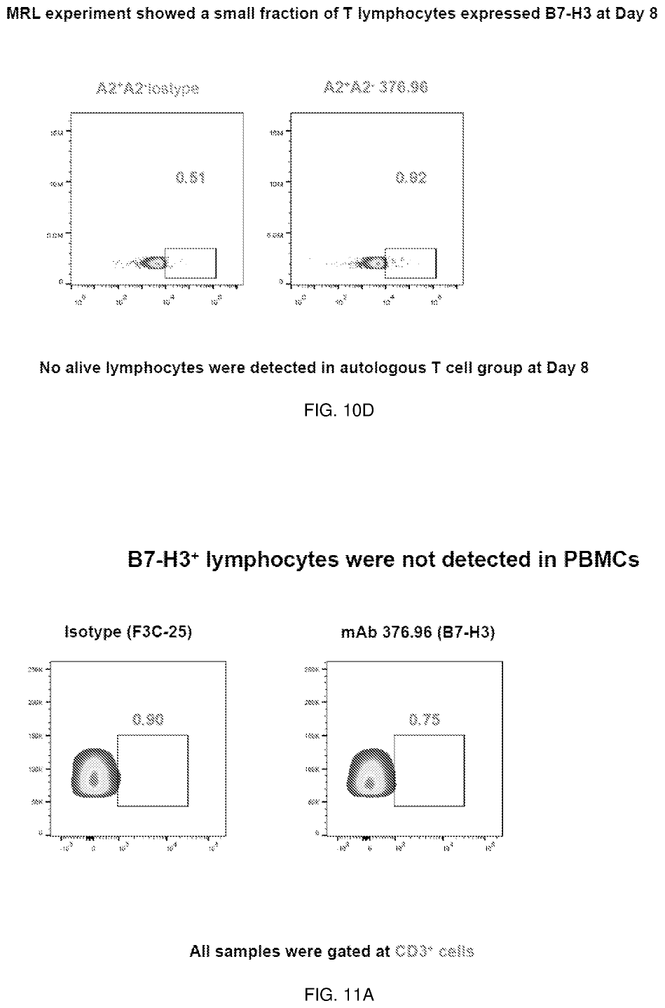

FIG. 10 shows that a small population of activated T cells expresses B7-H3 detected in mixed lymphocyte reaction (MLR). MLR is an in vitro assay using human leukocyte antigen (HLA) mismatched lymphocytes from two unrelated donors, which provokes T cell activation and an immune response against “non-self” cells. In this one-way MLR, the HLA-A2+ PBMC were used as responder cells and the HLA-A2− PMBC were irradiated (3Gy) as activator/stimulator cells. Both cells were mixed and co-cultured. On day 3, the responding lymphocytes underwent blast transformation and cellular proliferation in response to the HLA differences between the mixed two cell populations. The mixed autologous PBMC (A2−A2−) were used as a negative control (FIG. 10A). Increased activated CD3+ HLA-Class II+ T cells were detected in A2+A2− MLR (FIG. 10B) and CD3+ gated cells expressing B7-H3+ were detected by mAb 376.96 on days 5 (FIG. 10C) and 8 (FIG. 10D).

### B7-H3+ is Expressed on Peripheral Circulating T Cells and Infiltrated T Cells in the Liver Following Humanization of Immunodeficient NSG Mice (Xenogeneic GVHD Model)

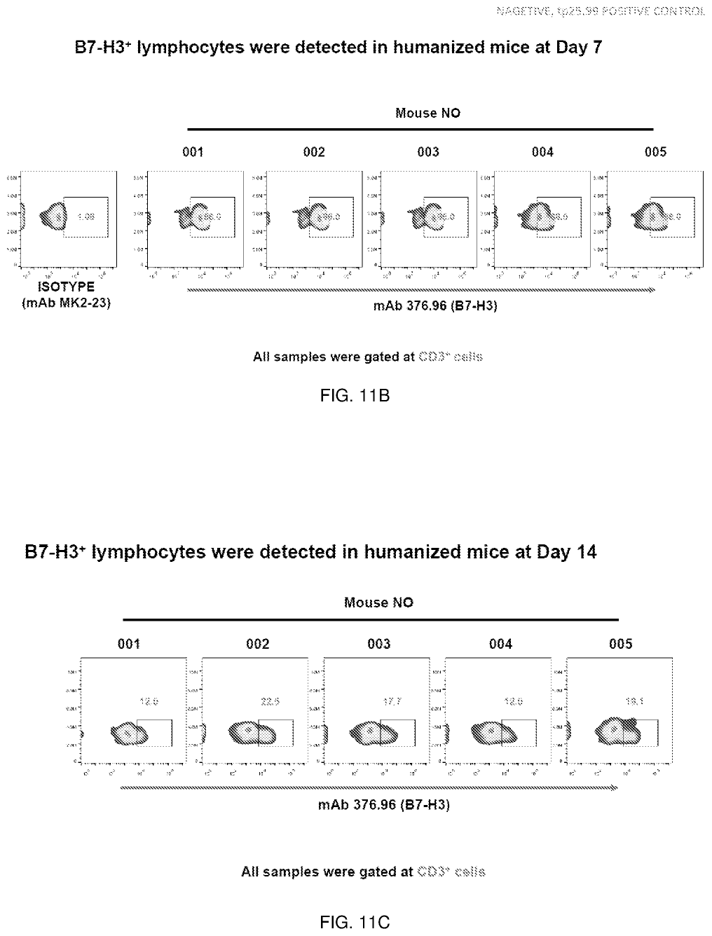

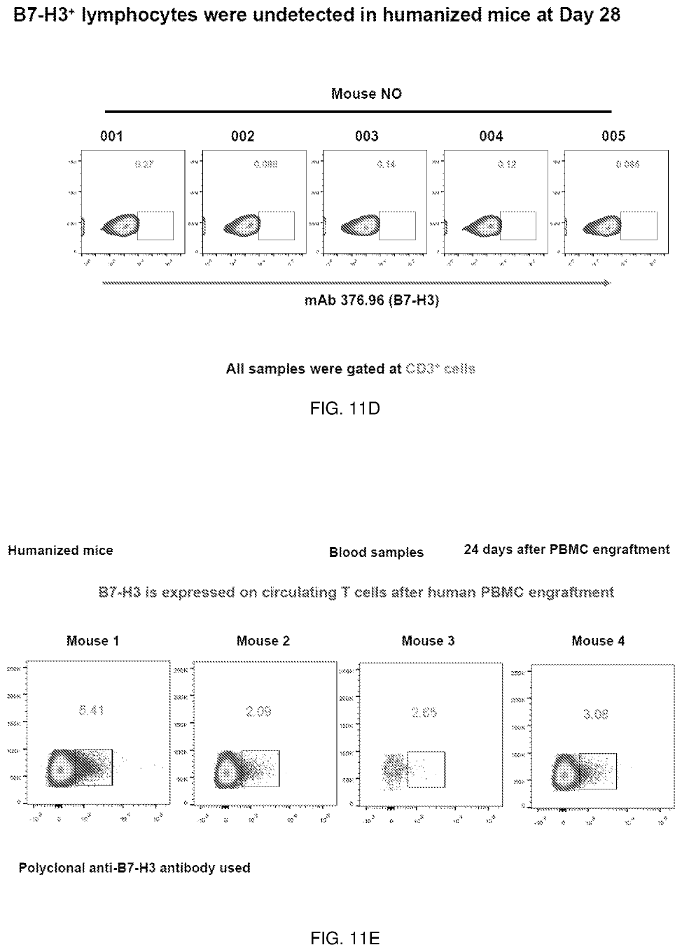

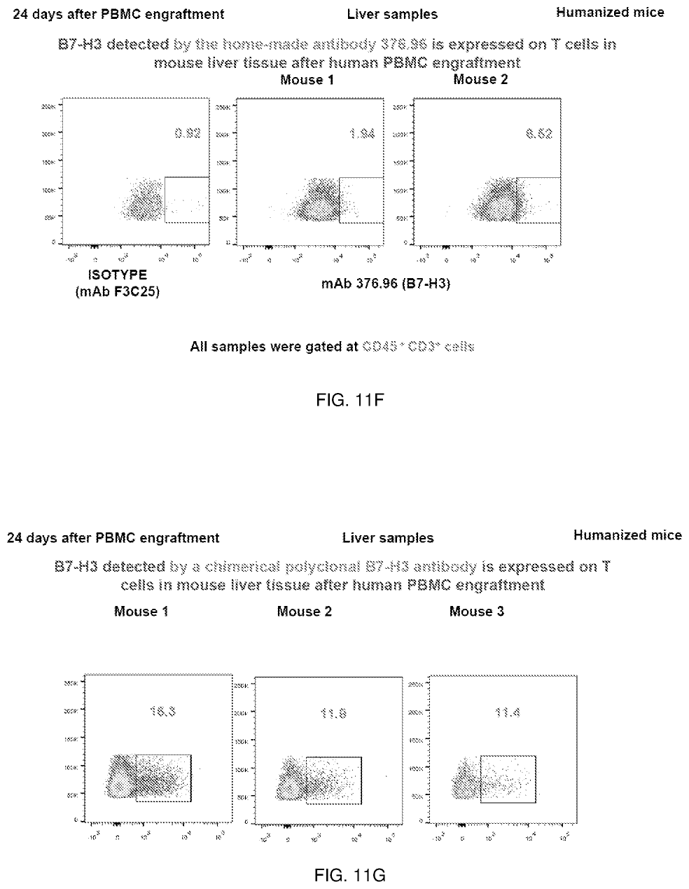

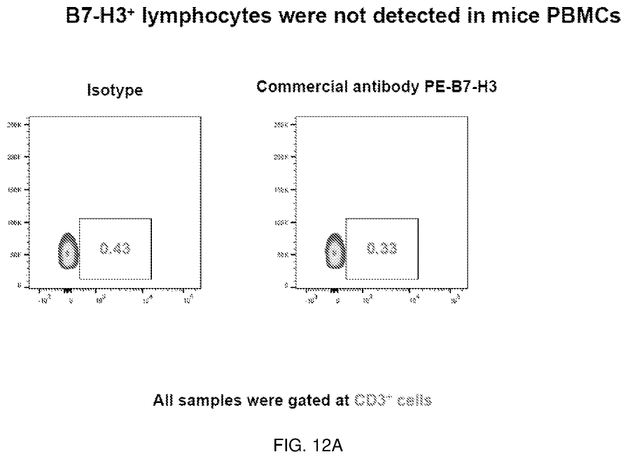

FIG. 11 shows that B7-H3+ is expressed on peripheral circulating T cells and infiltrated T cells in the liver following humanization of immunodeficient NSG mice (xenogeneic GVHD model). B7-H3 expression was not detected on human PBMC prior to in vivo transplantation (FIG. 11A). However, following xenotransplantation of these PBMC into NSG mice, B7-H3 was detected by mAb 376.96 on peripheral CD3+ T cells on days 7 (on peripheral circulating T cells in all humanized mice (n=5), ranging from 68% to 86% of CD3+ T cells) (FIG. 11B) and 14 (from 12% to 22.5%) (FIG. 11C) then became undetectable on day 28 (FIG. 11D). In addition, B7-H3 was detected at low levels by a polyclonal B7-H3 recognizing antibody on peripheral CD3+ T cells on day 24 (FIG. 11E). Moreover, B7-H3 was detected by mAb 376.96 (FIG. 11F) and the B7-H3 recognizing antibody (FIG. 11G) on mouse liver infiltrated CD3+ T cells on day 24. B7-H3 expression on CD3+ T cells was analyzed by flow cytometry.

### B7-H3 is Expressed on Peripheral Circulating T Cells in Allogeneic Mouse GVHD Models

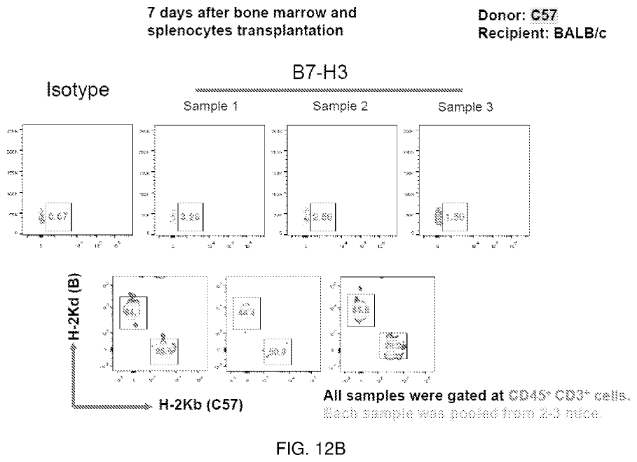

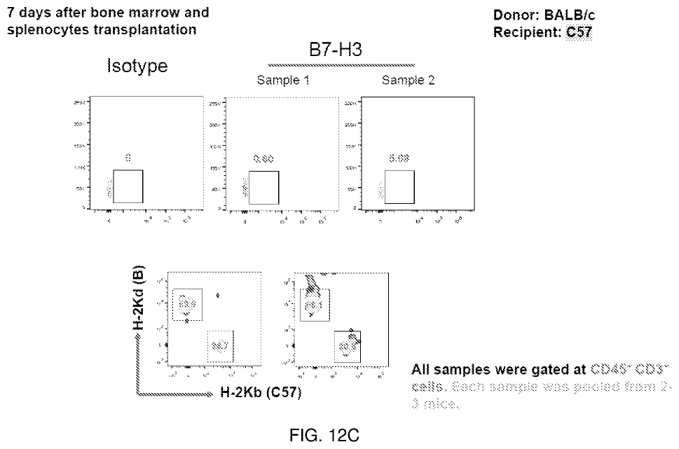

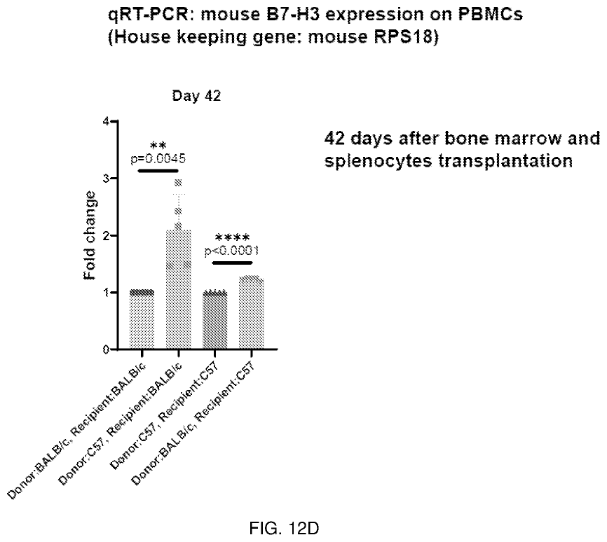

FIG. 12 shows that B7-H3 is expressed on peripheral circulating T cells in allogeneic mouse GVHD models. B7-H3 expression was not detected on mouse PBMC prior to in vivo transplantation (FIG. 12A). However, 7 days following allotransplantation of these PBMC into mice, B7-H3 was detected by a mouse B7-H3-specific antibody on donor (C57) peripheral CD3+ T cells in recipient mice (BLAB/c) (FIG. 12B) and donor (BALB/c) peripheral CD3+ T cells in recipient mice (BLAB/c) (FIG. 12C). In both cases donor chimerism of CD45+CD3+ was established in both models, detected by the presence of donor's MHC class I expression in the cells of peripheral blood of recipient mice (FIG. 12B,C). Although B7-H3 expression on T cells was not detectable by immunophenotypical analysis on day 42, B7-H3 mRNA in PBMC was higher in allogenic than syngeneic transplanted mice (FIG. 12D).

### Prevention/Treatment of Acute GVHD by B7-H3 CAR T Cells in Human PBMC Engrafted NSG Mice

Next, we conducted an additional set of in vivo experiments to i) confirm the data presented in FIG. 9 that B7-H3 CAR T cells can prevent/treat acute GVHD in 100% humanized mice (n=5, female NSG-(Kb Db) null (IA) null) with an increased number of NSG mice including both genders (n=10/group: n=5 female, n=5 male) and ii) test our hypothesis that merely inhibiting the immune checkpoint B7-H3 by mAb 376.96 therapy could worsen acute GVHD in the humanized mice.

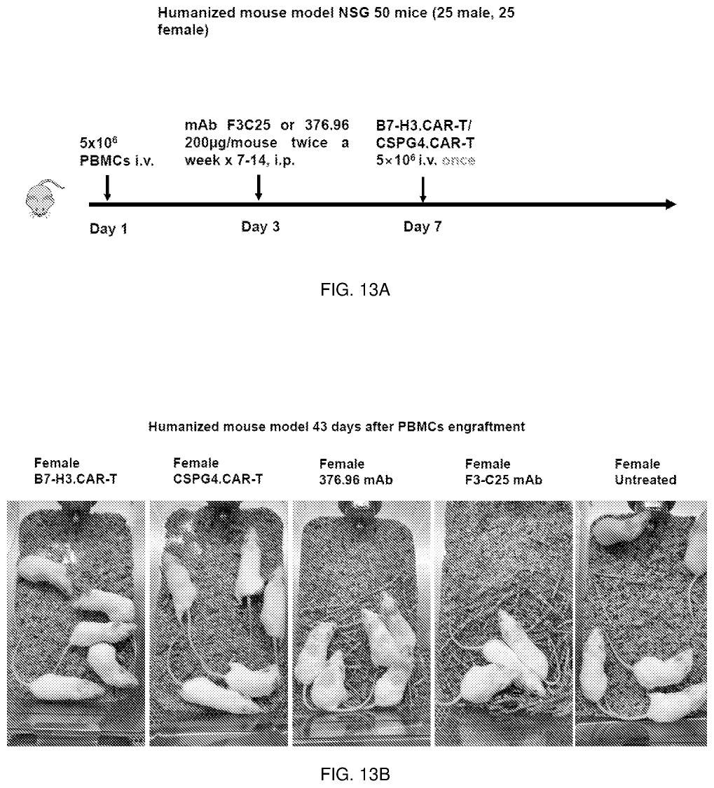

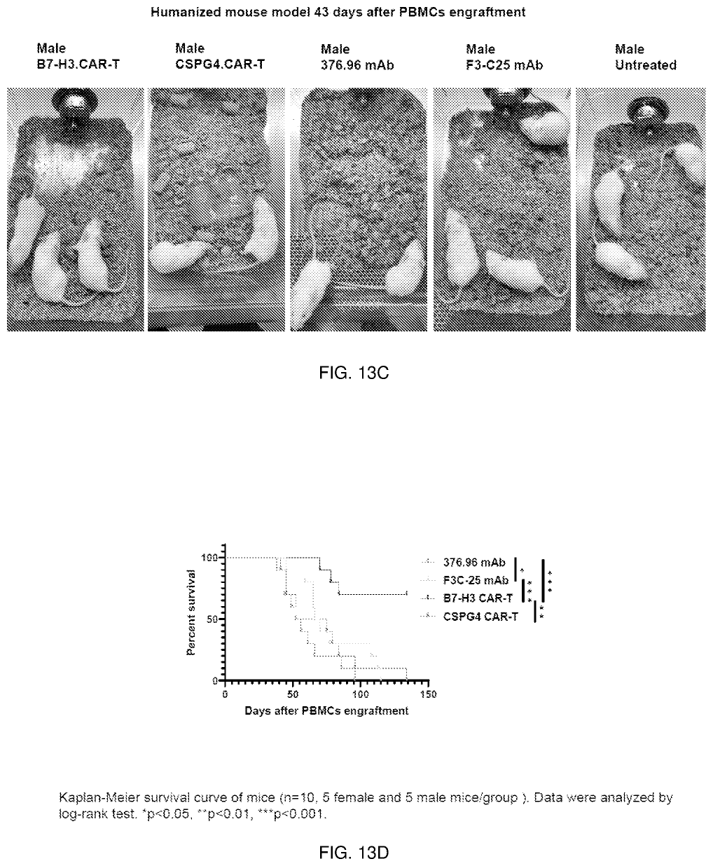

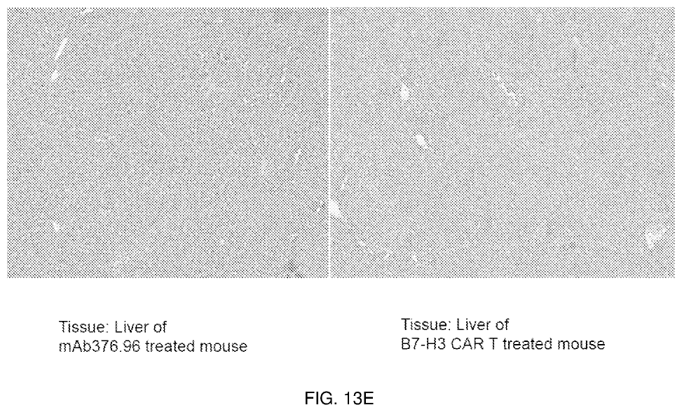

FIG. 13 shows the prevention/treatment of acute GVHD by B7-H3 CAR T cells in human PBMC engrafted NSG mice. Additional in vivo experiments testing the efficacy of B7-H3 CAR T cells vs B7-H3-specific mAb 376.96 were conducted in the xeno-GVHD (humanized NSG mice with human PBMC) mode model. The timeline and treatment are as indicated (FIG. 13A). The group of mice treated with mAb 376.96 was the first group among all groups that showed signs or symptoms of acute GVHD: fur loss (FIG. 13 B,C). Again, mice treated with B7-H3 CAR T cells have been having long-term survival. In contrast, mice treated with B7-H3-specific mAb 376.96 showed the worst survival. CSPG4 CAR T cells and the isotype control antibody F3C25 were used as specificity controls. Kaplan-Meier survival curve of mice (n=10, 5 female and 5 male mice/group). Data were analyzed by log-rank test. *p<0.05, **p<0.01, ***p<0.001 (FIG. 13D). H&E stained liver samples collected after death from mice treated with mAb 376.96 showed the most severe inflammation, while the liver tissue collected from the mice treated with B7-H3 CAR T cells had the mildest degree of inflammation (FIG. 13E).

### Conclusions

B7-H3 targeting chimeric antigen receptor (CAR) T cells prevented GvHD in 100% humanized mice.

In human PBMC humanized NSG-(Kb Db) null (IAnull) mice, all developed and died from GVHD (15% body weight loss, hunched posture, fur loss and reduced mobility), except for the group of mice treated with B7-H3 CAR T cells. The B7-H3 CAR T cells derived from the same donor PBMC treated and survived 5 mice have no sign of GVHD.

B7-H3 CAR T cells did not eliminate the engrafted human PBMC cells. Instead, long-term survival of human PBMC grafts including T lymphocytes was found in all B7-H3 CAR T cells treated humanized NSG-(Kb Db) null (IAnull) mice.

B7-H3 CAR T cell therapy effectively prevents/treats xenogeneic acute GVHD (human-mouse) in both NSG-(Kb Db) null (null) (5 female) and NSG mice (10 mice including 50% of male and female mice) by killing activated donor T cells expressing the inhibitory immune checkpoint B7-H3. Thus, taking advantage of inhibitory immune checkpoint upregulation on activated donor T cells following HSCT, specific CAR T cells targeting these checkpoints can eliminate activated T cells responsible for acute GVHD.

The B7-H3 monoclonal antibody 376.96 worsened murine acute GVHD when given after HSCT-reflecting the fact that B7-H3 is an inhibitory immune checkpoint, and inhibition would result in additional immune activation.

### Use

In one example, it is estimated that over 20,000 allogeneic HSCT procedures are carried out annually worldwide with slow but steady growth. All patients undergoing allogeneic HSCT receive some form of prophylaxis to prevent acute GVHD. 30-60% of patients after HSCT will require systemic therapy for acute GVHD with 50% of these patients requiring 2nd line therapy. Donor-derived B7-H3 directed CAR T cell therapy or other inhibitory immune checkpoint (such as PD-1/PD-L1, TIM-3, or B7-H4) directed CAR T therapy represents a highly selective and non-systemic immune-suppressive approach to preventing and treating GVHD.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations described herein following, in general, the principles described herein and including such departures from the invention that come within known or customary practice within the art to which the invention pertains and may be applied to the essential features hereinbefore set forth, and follows in the scope of the claims.

Other embodiments are within the claims.

What is claimed is:

1. A method for treating graft versus host disease (GVHD) in a human, comprising administering to said human a therapeutically effective amount of cells each of which comprises a chimeric antigen receptor (CAR) which is specifically directed against an immune checkpoint molecule.

2. The method of claim 1, wherein said cells are produced using immune cells obtained from a donor of cells or tissue which is to be transplanted.

3. The method of claim 1, wherein GVHD comprises acute GVHD.

4. The method of claim 1, wherein the immune checkpoint molecule is B7-H3, PD-1/PD-L1, TIM-3, or B7-H4.

5. The method of claim 1, wherein the cells are T lymphocytes.

6. The method of claim 1, wherein the CAR is specifically directed against B7-H3.

7. The method of claim 6, wherein said CAR comprises variable regions of the heavy and light chains of the 376.96 mAb.

8. The method of claim 1, wherein the chimeric antigen receptor comprises a human CD8a hinge and transmembrane domain, CD28 or 4-1BB intracellular costimulatory domains, and CD3z intracellular signaling domain.

9. The method of claim 1, wherein the cells are administered intravenously.

10. The method of claim 1, wherein the cells are administered within 24 hours after a transplantation procedure.

11. A method of treating allogeneic tissue that is to be transplanted into a human, comprising contacting the tissue with cells each of which comprises a chimeric antigen receptor (CAR) which is specifically directed against an immune checkpoint molecule.

12. The method of claim 11, wherein the allogeneic tissue comprises cells.

13. The method of claim 11, wherein the immune checkpoint molecule is B7-H3, PD-1/PD-L1, TIM-3, or B7-H4.

14. The method of claim 11, wherein the cells are T lymphocytes.

15. The method of claim 11, wherein the CAR is specifically directed against B7-H3.

16. A method for treating graft versus leukemia in a human, comprising administering to said human a therapeutically effective amount of cells each of which comprises a chimeric antigen receptor (CAR) which is specifically directed against an immune checkpoint molecule.

17. The method of claim 16, wherein said cells are produced using immune cells obtained from a donor of cells or tissue which is to be transplanted.

18. The method of claim 16, wherein the immune checkpoint molecule is B7-H3, PD-1/PD-L1, TIM-3, or B7-H4.

19. The method of claim 16, wherein the cells are T lymphocytes.

20. The method of claim 16, wherein the CAR is specifically directed against B7-H3.

21. The method of claim 20, wherein said CAR comprises variable regions of the heavy and light chains of the 376.96 mAb.

22. The method of claim 16, wherein the chimeric antigen receptor comprises a human CD8a hinge and transmembrane domain, CD28 or 4-1BB intracellular costimulatory domains, and CD3z intracellular signaling domain.

23. The method of claim 16, wherein the cells are administered intravenously.

24. The method of claim 16, wherein the cells are administered within 24 hours after a transplantation procedure.
